(19) United States
(12) Patent Application Publication
(10) Pub. No.: US 2009/0054526 A1
(43) Pub. Date: Feb. 26, 2009

(54) INHIBITORS OF ANOREXIC LIPID HYDROLYSIS FOR THE TREATMENT OF EATING DISORDERS

(76) Inventors: Harald S. Hansen, Vanlose (DK); Gitte Petersen, Lyngby (DK); Andreas Artmann, Smorum (DK); Philip J. Larsen, Virum (DK)

Correspondence Address: DARBY & DARBY P.C., P.O. BOX 770, Church Street Station, New York, NY, 10008-0770, US

(21) Appl. No.: 11/995,401

(22) PCT Filed: Jul. 14, 2006
(86) PCT No.: PCT/DK06/50030

§ 371 Date: May 29, 2008

Related U.S. Application Data
(60) Provisional application No. 60/699,983, filed on Jul. 14, 2005.

(30) Foreign Application Priority Data
Jul. 14, 2005 (DK) PA200501029

(51) Int. Cl. A61K 31/165; A61P 3/04; A61P 1/00

(52) U.S. Cl. 514/625

(57) ABSTRACT

Compounds, pharmaceuticals, cosmetic or dietary supplements for the treatment of overweight, obesity and/or type II diabetes in a mammal (e.g. human) comprising a compound with formula I or formula II for example ceramidase-inhibitor, such as (1S,2R)-D-ery-thro-2-(N-myristoylamino)-1-phenyl-1-propanol, alone or in combination with an anorexic lipid (or other appetite-inhibiting acylamides or oleoyl-estrone), and methods of treatment comprising administration of said compounds, pharmaceuticals, cosmetic or a dietary supplements. The compounds, pharmaceuticals, cosmetic or dietary supplements and methods of the invention may further be used in modifying the feeding behaviour, suppression of hunger, enhancement of satiety, reduction of energy intake, reduction of fat tissue mass/lean mass ratio in a mammal (e.g. human).

### BACKGROUND OF THE INVENTION

Obesity is associated with numerous health risks, which range from non-fatal debilitating conditions such as osteoarthritis, to life-threatening chronic diseases such as coronary heart disease, diabetes type II and certain types of cancer. The physiological consequences of obesity can range from lowered self-esteem to clinical depression (1). The prevalence of obesity is increasing in both developed and undeveloped countries in an epidemic fashion (2). Since dietary therapy often has a low success rate in the long term, there has been an increasing demand for pharmaceutical alternatives and a large number of different drug targets have been suggested (1-3). Both decreasing nutrient absorption, inhibiting appetite as well as increasing thermogenesis are being considered, all of which have drawbacks. Decreasing nutrient absorption, for example by inducing fat mal-absorption may affect gastrointestinal functions and cause gastrointestinal discomfort. Inhibiting appetite is generally thought to involve targeting brain structures with for example peptide analogues that may have difficulty reaching their target in the brain. Furthermore, drugs targeted for the brain will lead to exposure of non-target tissues, independently of whether they are administered orally or subcutaneously, thereby causing potentially unwanted side effects. Increasing thermogenesis may affect different hormonal mechanisms that may have serious side effects in the long term. A compound naturally occurring in the human diet that will decrease appetite via a direct pharmacological/physiological action on the intestine is a desirable candidate for appetite regulation.

Oleoylethanolamide is a naturally occurring compound found in plants and mammals (4) that is called anorexic lipid (5). The endogenous level of oleoylethanolamide in the intestine displays diurnal fluctuations in response to nutrient status (6). It is believed to regulate food intake, since oral (7, 8) as well as intraperitoneal administration of oleoylethanolamide inhibits food intake in rodents (6). Oleoylethanolamide may execute its anorexic effect through activation of the transcription factor PPARα (peroxisome-proliferator activated receptor-alpha) locally in the intestine, which may in turn inhibit feeding via activation of vagal c-fibers that engage brain structures such as the nucleus solitary tract in the brainstem and paraventricular nucleus in the hypothalamus (6, 9). Palmitoylethanolamide and elaidoylethanolamide also inhibit food intake following intraperitoneal injections, although they are slightly less potent than oleoylethanolamide (5). Oleoyl-estrone is another anorexic lipid (10, 11), and it is likely that its mode of action is via activation of PPARα. Although oral administration of such appetite-inhibiting acylamides is proposed for treating obesity (WO02/080860) their rapid degradation in the gastrointestinal system (7) reduces their bioavailability and therapeutic effect. Thus, there remains a need to provide improved methods, compounds and pharmaceutical and cosmetic compositions for the control of dietary intake and obesity.

### SUMMARY OF THE INVENTION

Several studies have shown oleoylethanolamide (OEA) to be an inhibitor of appetite when administered either orally or by intraperitoneal injection. However, the appetite-reducing effects of exogenous (and probably also endogenous) OEA are limited due to its very short intestinal half-life. The molecular mechanisms that control OEA turnover in response to feeding and diurnal cycles have not been characterised (6). Turnover of OEA involves its enzymatic hydrolysis to oleic acid and ethanolamine degradation. OEA is a substrate for the two N-acylethanolamine hydrolases, fatty acid amide hydrolase (FAAH) (12) and N-palmitoylethanolamine-hydrolyzing acid amidase (NPAA) (13). NPAA, in particular, has been considered a likely candidate for control of OEA turnover since it is abundant in the small intestine (14).

In contrast to current theories and data regarding OEA turnover (6), the present invention is based on the theory that the enzyme such as ceramidase mediates OEA degradation and turnover in the intestine, and that OEA catabolism by such intestinal hydrolases provides a mechanism indirectly controlling the appetite regulating effects of OEA. Although, acid ceramidase is known to be inhibited by OEA (15), a role for this enzyme in OEA catabolism or appetite regulation has never previously been suggested. While enzymes such as ceramidases are known to degrade ceramides (15-16), it is now proposed to play a role in the degradation of oleoylethanolamide, and possibly oleoyl-estrone. A novel approach to controlling levels of OEA, and hence appetite regulation, is for example to reduce the rate of OEA hydrolysis by providing compounds and pharmaceutical compositions comprising inhibitors of ceramidase activity. Inhibition of ceramidase activity may improve both the bioavailability of exogenously administered anorexic lipids as well as potentiate the effect of exogenously administered and endogenously produced anorexic lipids such as oleoylethanolamide, palmitoylethanolamide, elaidoylethanolamide and also oleoyl-estrone. In particular the present invention provides compounds and pharmaceutical compositions comprising a compound with formula I or II having the properties of an appetite suppressant for example a ceramidase inhibitor; alone or in combination with a further appetite suppressant compound, for example exogenous anorexic lipid, which may be used to reduce appetite and thereby provide a treatment for obesity and obesity-related diseases.

In one embodiment, the invention is directed to the use of a compound with formula I or II for example ceramidase inhibitor for the manufacture of a pharmaceutical composition for the prophylaxis or therapeutic treatment of diseases or disorders associated with impaired appetite regulation in a mammal, wherein said compound is an appetite suppressing or satiety inducing agent and has the formula I:

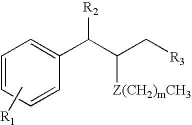

wherein m is an integer ranging from 0 to 22; Z is a member selected from —C(O)N(R₄)—; —(R₄)NC(O)—; —OC(O)—; —(O)CO—; O; NR₄; and S; and
R₁, R₂, R₃, and R₄ are independently selected from the group consisting of substituted or unsubstituted alkyl, hydrogen, NO₂, OH, methoxy, chlorine, bromine, fluorine, substituted or unsubstituted C₁-C₆ alkyl, substituted or unsubstituted lower (C₁-C₆) acyl, ether, homoalkyl, and aryl. Up to twelve hydrogen atoms of the compound may also be substituted by a methyl group, a double bond, or a triple bond.

In another embodiment, the invention is directed to the use of a compound with formula I or II for example ceramidase inhibitor for the manufacture of a pharmaceutical composition for the prophylaxis or therapeutic treatment of diseases or disorders associated with impaired appetite regulation in a mammal, wherein said compound is an appetite suppressing or satiety inducing agent and has the formula II:

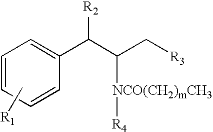

wherein m is an integer ranging from 6 to 18; R₁, R₂, R₃, and R₄ are independently selected from the group consisting of substituted or unsubstituted alkyl, hydrogen, NO₂, OH, methoxy, chlorine, bromine, fluorine, substituted or unsubstituted C₁-C₆ alkyl, substituted or unsubstituted lower (C₁-C₆) acyl, ether, homoalkyl, and aryl. Up to twelve hydrogen atoms of the compound may also be substituted by a methyl group, a double bond, or a triple bond.

In an alternative embodiment, the invention is directed the use of a composition comprising a compound with formula I or II for example a ceramidase inhibitor for non-therapeutic administration to a mammal as an appetite suppressant in a dosage sufficient to effect appetite suppression and repeating said dosage until a cosmetically beneficial loss of body weight has occurred, wherein said compound has the formula I:

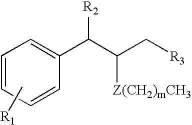

wherein m is an integer ranging from 0 to 22; Z is a member selected from —C(O)N(R₄)—; —(R₄)NC(O)—; —OC(O)—; —(O)CO—; O; NR₄; and S; and
R₁, R₂, R₃, and R₄ are independently selected from the group consisting of substituted or unsubstituted alkyl, hydrogen, NO₂, OH, methoxy, chlorine, bromine, fluorine, substituted or unsubstituted C₁-C₆ alkyl, substituted or unsubstituted lower (C₁-C₆) acyl, ether, homoalkyl, and aryl. Up to twelve hydrogen atoms of the compound may also be substituted by a methyl group, a double bond, or a triple bond.

In an alternative embodiment, the invention is directed a method for treatment of overweight, obesity and/or type II diabetes, the method comprising administering to a human or a domestic animal in need thereof an effective amount of a compound with formula I for example a ceramidase inhibitor, wherein said compound is an appetite suppressing or satiety inducing agent with the following structure:

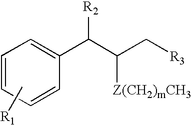

wherein m is an integer ranging from 0 to 22; Z is a member selected from —C(O)N(R₄)—; —(R₄)NC(O)—; —OC(O)—; —(O)CO—; O; NR₄; and S; and
R₁, R₂, R₃, and R₄ are independently selected from the group consisting of substituted or unsubstituted alkyl, hydrogen, NO₂, OH, methoxy, chlorine, bromine, fluorine, substituted or unsubstituted C₁-C₆ alkyl, substituted or unsubstituted lower (C₁-C₆) acyl, ether, homoalkyl, and aryl. Up to twelve hydrogen atoms of the compound may also be substituted by a methyl group, a double bond, or a triple bond.

In a further embodiment, the invention is directed to a solid composition for use as a medicament comprising compound with formula I for example a ceramidase inhibitor with the formula I:

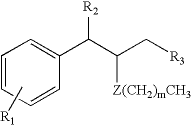

wherein m is an integer ranging from 0 to 22; Z is a member selected from —C(O)N(R₄)—; —(R₄)NC(O)—; —OC(O)—; —(O)CO—; O; NR₄; and S; and R₁, R₂, R₃, and R4 are independently selected from the group consisting of substituted or unsubstituted alkyl, hydrogen, NO₂, OH, methoxy, chlorine, bromine, fluorine, substituted or unsubstituted C₁-C₆ alkyl, substituted or unsubstituted lower (C₁-C₆) acyl, ether, homoalkyl, and aryl, and further comprising one or more appetite suppressing compounds with the formula:

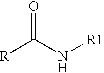

wherein R—C═O is derived from a natural or synthetic fatty acid and R1 is i) a branched or unbranched, saturated or unsaturated, substituted or unsubstituted chain of from 1 to 30 carbon atoms, which optionally is substituted with one or more hydroxy groups, which may be primary, secondary or tertiary, or ii) an N-terminal amino acid or peptide residue, together with a pharmaceutically acceptable excipient.

### BRIEF DESCRIPTION OF THE DRAWINGS

FIG. 1. Structures of (1S,2R) D-erythro-N-myristoyl-2-amino-1-phenyl-1-propanol (D-erythro-MAPP), (1R,2S) L-erythro-N-myristoyl-2-amino-1-phenyl-1-propanol (L-erythro-MAPP), (1R,2R) D-threo-N-myristoyl-2-amino-1-(4′-nitrophenyl)-1,3-propandiol (D-threo-NMAPPD=B13), (1S,2S) L-threo-N-myristoyl-2-amino-1-(4′-nitrophenyl)-1,3-propandiol (L-threo-NMAPPD), and (2S,3R,4E) D-erythro-ceramide. The Fisher projections of D-erythro-MAPP, D-threo-NMAPPD, and D-erythro-ceramide are also shown. In absolute configuration, D-erythro-ceramide corresponds to L-erythro-MAPP (D-erythro-MAPP having the enantiomeric configuration) and L-erythro-NMAPPD (D-threo-NMAPPD having a diastereomeric configuration).

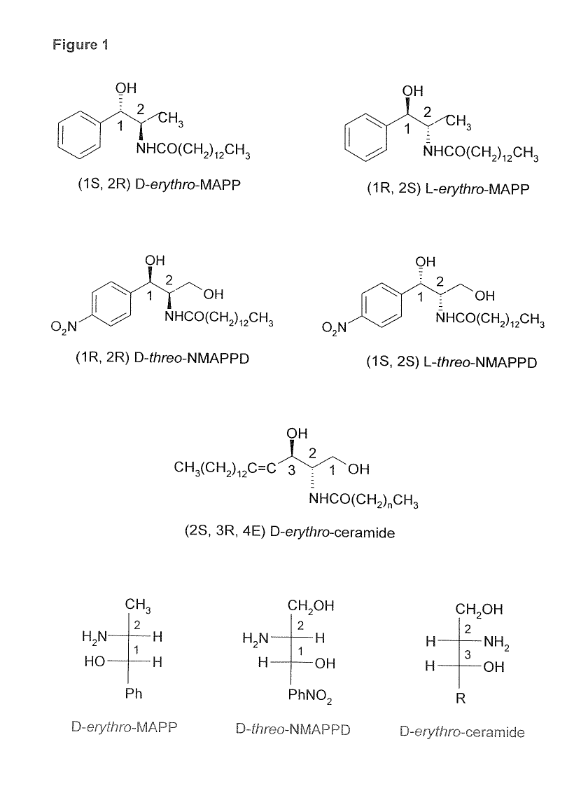

FIG. 2. Effect of increasing concentrations of the FAAH inhibitor URB597 (A), the ceramidase inhibitor D-erythro-MAPP (B), and the ceramidase inhibitor D-threo-NMAPPD (C) on rate of hydrolysis in rat intestinal homogenate (25 μg of protein) utilizing 50 μM of substrate in a total volume of 100 μl containing 100 mM citrate phosphate (pH 7.0) and 8 mM CHAPS and 20 min of incubation at 37° C. ¹⁴C-Octanoyl-D-sphingosine (ceramide; 25.000 dpm), ³H-oleoylethanolamide (OEA; 25.000 dpm), and ³H-anandamide (AEA; 25.000 dpm) were used as substrates. Results represent in A 1 experiment, in B 2 experiments, and in C₂₋₄ experiments each performed in duplicate.

FIG. 3. Effect of pH on rate of hydrolysis of ceramide, OEA, and AEA in rat intestinal homogenate (A-B) and adding 10 μM of the FAAH inhibitor URB597 (C). Ceramide (¹⁴C-octanoyl-D-sphingosine), OEA (³H-oleoylethanolamide), and AEA (³H-anandamide) were utilized as substrates (50 μM) and 25 μg of protein was added in a total volume of 100 μl followed by 20 min incubation at 37° C. Buffers were used in 100 mM concentration with 8 mM of CHAPS: Citrate-NaHPO₄ (pH 4.0-7.0), tris-HCl (pH 7.0-9.0), and glycine-NaOH (pH 9.0-10.5). Results in A (B) represent n=2 (n=4-7) independent experiments each performed in duplicate (mean±SEM) while results presented in C are from one experiment performed in duplicate.

FIG. 4. Sub-chronic effects on (A) food intake, (B) water intake, and (C) body weight in dietary obese mice following daily i.p. administration of OEA and/or D-threo-NMAPPD in vehicle (just before onset of dark) depicted in cumulative bar graphs. Values are mean±SEM (n=10 except for vehicle group in which n=9). * (OEA 2 mg/kg), ^{¤}(OEA 5 mg/kg), “(D-threo-NMAPPD 30 mg/kg), and ⁺(OEA 2 mg/kg+D-threo-NMAPPD 30 mg/kg): P<0.05 using one-way ANOVA followed by Fisher's post hoc test.

### DETAILED DISCLOSURE OF THE INVENTION

The present invention provides a composition, pharmaceutical preparation, cosmetic or dietary supplement comprising a compound with the chemical structure of formula I or II, which is an appetite suppressing or satiety inducing agent, either alone or in combination with one or more fatty acid alkanolamide compound, homologue, or analogue for use in a treatment to reduce body weight, obesity and/or type II diabetes and other obesity associated diseases, such as coronary heart disease in a mammal (human or domestic animal). In one embodiment, said compound of formula I or II is an inhibitor, for example a ceramidase inhibitor, homologue, or analogue thereof.

The invention also provides methods for reducing food intake in a mammal (e.g. a human and/or a domestic animal) in need thereof, by administration of said pharmaceutical preparation, cosmetic or dietary supplement comprising a compound with chemical structure of formula I or II, either alone or in combination with a fatty acid alkanolamide compound, homologue, or analogue, in an amount/amounts sufficient to reduce body fat, body weight or prevent body fat or body weight gain. In one embodiment, said compound is an inhibitor, for example a ceramidase inhibitor, homologue, or analogue thereof.

### DEFINITIONS

The term “pharmaceutical composition” indicates a composition suitable for pharmaceutical use in a subject, including an animal or human. A pharmaceutical composition generally comprises an effective amount of an active agent and a pharmaceutically acceptable carrier.

Compounds of the invention may contain one or more asymmetric centres and can thus occur as racemates and racemic mixtures, single enantiomers, diastereomeric mixtures and individual diastereomers. The invention comprehends all such isomeric forms of the active compounds of the invention.

Where substituent groups are specified by their conventional chemical formulae, written from left to right, they equally encompass the chemically identical substituents which would result from writing the structure from right to left, e.g. —CH₂O— is intended to also recite —OCH₂—.

Compounds of the invention that contain olefinic double bonds, and unless otherwise specified, are meant to include both E and Z geometric isomers. Compounds of the invention may exist as tautomers, with different points of attachment of hydrogen, such as ketone and its enol form, known as keto-enol tautomers. The individual tautomers, as well as a mixture thereof, are encompassed by the claimed inventive compounds.

Compounds of the invention include diastereoisomers of pairs of enantiomers. Said diastereomers can for example be obtained by fractional crystallisation from a suitable solvent, e.g. methanol or ethyl acetate or a mixture thereof, and the pairs of enantiomers obtained are then separated into individual stereoisomers by conventional methods, e.g. by use of a resolving agent such as an optically active acid. Any enantiomer of the compounds of the invention can also be obtained by stereospecific synthesis, using optically pure starting materials or reagents of known configuration.

The term “heteroatom” is meant to include oxygen (O), nitrogen (N), sulphur (S) and silicon (Si).

The term “alkanol” refers to a saturated or unsaturated, substituted or unsubstituted, branched or unbranched alkyl group having a hydroxyl substituent, or a substituent derivable from a hydroxyl moiety, e.g. ether, ester. Also alkanol substituted with a nitrogen-, sulphur-, or oxygen-bearing substituent that is included in bond Z, between the fatty acid and the phenylalkanol.

The term “fatty acid” refers to a saturated or unsaturated substituted or unsubstituted, branched or unbranched alkyl group having a carboxyl substituent, and further includes species in which the carboxyl substituent is replaced with a —CH₂— moiety. Examples of fatty acids of the invention are C₄-C₂₂ acids.

In the present context, the term “alkyl” by itself, or as part of another substituent, is intended to indicate a branched or straight-chain, or cyclic hydrocarbon radical, or combination thereof, which may be fully saturated, mono- or polyunsaturated and can include di- and multivalent radicals, having the number of carbon atoms designated (i.e. C₁-C₁₀). Examples of saturated hydrocarbon radicals include groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of n-pentyl, n-hexyl, n-heptyl, n-octyl.

The term “alkenyl” is intended to indicate an unsaturated alkyl group having one or more double bonds or triple bonds, for example vinyl, 2-propenyl, crotyl, 2-isopentyl, 2-(butadienyl), 2,4,-pentadienyl, 3-(1,4-pentadienyl), ethynyl, 1- and 3-propynyl, 3-butynyl, and higher homologues and isomers.

The term “alkylene” by itself or as part of another substituent means a divalent radical derived from an alkane, as exemplified by —CH₂CH₂CH₂ CH₂—, and includes groups described as “heteroalkylene”. An alkyl (or alkylene) group of the invention may have between 1 to 24 carbon atoms.

The terms “alkoxy”, “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl”, by itself or in combination with another term means a stable straight or branched chain, or cyclic hydrocarbon radical, or combinations thereof, consisting of the stated number of carbon atoms and at least one heteroatom selected from the group consisting of O, N, Si and S, and wherein the nitrogen and sulphur atoms may optionally be oxidised and the nitrogen heteroatom may optionally be quaternized. The heteroatom(s) O, N, and S, and Si may be placed at any interior position of the heteroalkyl group or at the position at which the alkyl group is attached to the remainder of the molecule, for example: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, as for example —CH₂—NH—OCH₃, and —CH₂—O—Si(CH₃)₃. Similarly, the term “heteroalkylene” by itself or as part of another substituent means a divalent radical derived from heteroalkyl, as for example —CH₂—CH₂—S—CH₂—CH₂, and —CH₂—S—CH₂—CH₂—NH—CH₂. In the case of heteroalkylene groups, heteroatoms can also occupy either or both of the chain termini (for example alkyleneoxy, alkylenedioxy, alkyleneamino, and alkylenediamino).

The terms “cycloalkyl” and “heterocycloalkyl” by themselves or in combination with other terms, represent cyclic versions of “alkyl” and “heteroalkyl” respectively. Furthermore, in a heterocycloalkyl, a heteroatom can occupy the position at which the heterocycle is attached to the remainder of the molecule. A cycloalkyl may be a cyclopentyl, cyclohexyl, 1-cyclohexenyl, 3-cyclohexenyl, and cycloheptyl. A heterocycloalkyl may be 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, and 2-piperazinyl.

The terms “halo” or “halogen”, by themselves or as part of a substituent, mean a fluorine, chlorine, bromine, or iodine atom. The term “haloalkyl” is meant to include monohaloalkyl and polyhaloalkyl, and the term “halo(C₁-C₄)alkyl” is meant to include trifluoromethyl; 2,2,2-trifluoroethyl; 4,chlorobutyl and 3-bromopropyl.

The term “aryl” means a polyunsaturated, aromatic hydrocarbon substituent which can be a single ring or multiple rings (from 1 to 3 rings) fused together or linked covalently. The term “heteroaryl” refers to aryl groups (or rings) that contain from one to four heteroatoms selected from N, O, and S, wherein the nitrogen and sulphur atoms are optionally oxidised, and the nitrogen atom(s) are optionally quaternized. A heteroaryl group can be attached to the remainder of the molecule through a heteroatom. Examples of an aryl or heteroaryl group include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents of any one of these aryl and heteroaryl ring systems are selected from the group of acceptable substituents given below.

The term “arylalkyl” refers to radicals in which an aryl group is attached to an alkyl group, such as benzyl, phenethyl, and pyridylmethyl, and includes those alkyl groups in which a carbon atom, such as a methylene group, has been replaced by for example an oxygen atom, as in phenoxymethyl, 2-pyridyloxymethyl, and 3-(1-naphthloxy)propyl. The terms alkyl, heteroalkyl, aryl and heteroaryl each include both substituted and unsubstituted forms of the indicated radical. Examples for each type of radical are given below. Substituents of the alkyl and heteroalkyl radicals (including the groups alkylene, alkenyl, heteroalkylene, heteroalkenyl, alkynyl, cycloalkyl, heterocycloalkyl, cycloalkenyl, and heterocycloalkenyl) can be one or more of a variety of groups selected from: —OR′, ═O, ═NR′, ═N—OR′, ═NR′R″, —SR′, halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R“ ” each independently refer to hydrogen, substituted or unsubstituted heteroalkyl, substituted or unsubstituted aryl, for example aryl substituted with 1-3 halogens, substituted or unsubstituted alkyl, alkoxy or thioalkoxy groups, or arylalkyl groups. A compound of the invention that comprises more than one R group, for example, each of the R groups is independently selected as are each R′, R″, R′″ and R“ ” groups when more than one of these is present. Where R′ and R″ are attached to the same nitrogen atom, they may be combined with the nitrogen atom to form a 5-, 6-, or 7-membered ring. Thus -NR′R″ may be 1-pyrrolidinyl and 4-morpholinyl. With respect to the above substituents, the term, “alkyl” includes carbon atoms bound to groups other than hydrogen groups, such as haloalkyl (e.g. —CF₃ and —CH₂CF₃) and acyl (e.g. —C(O)CH₃, —C(O)CF₃, —C(O)CH₂OCH₃).

Substituents of the aryl and heteroaryl groups may include halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN, —NO₂, —R′, —N₃, —CH(Ph)₂, fluoro (C₁-C₄) alkoxy, and fluoro (C₁-C₄) alkyl, in a number ranging from zero to the total number of open valences on the aromatic ring system; and where R′, R″, R′″ and R″″ are independently selected from hydrogen, (C₁-C₈)alkyl and heteroalkyl, unsubstituted aryl and heteroaryl, (unsubstituted aryl)-(C₁-C₄),alkyl, and (unsubstituted aryl)oxy-(C₁-C₄)alkyl. When a compound of the invention includes more than one R group, for example, each of the R groups is independently selected as are each R′, R″, R′″ and R“ ” groups when more than one of these is present.

The term “fatty acid oxidation” refers to the conversion of fatty acids into ketone bodies.

The term “modulate” means to induce a change wherein a modulator of ceramidase activity decreases the rate of fatty acid oxidation.

The term “OEA” is an abbreviation for oleoylethanolamide, which is a natural lipid.

The term “weight loss” refers to a loss of a portion of total body weight.

The term “effective amount” or “sufficient dosage” is one that is required to produce a desired result in terms of the subjective or objective improvement of the recipient of treatment. The subjective improvement may be measured in terms of appetite suppression and an objective improvement may be measured in terms of one or more of the following parameters: loss of body weight, body fat, decreased food consumption, decreased food seeking behaviour, improved serum lipid profile, decreased likelihood of developing a disease or harmful health condition.

A “prophylactic treatment” is one that is administered to a subject (mammal), who does not exhibit signs of a disease, or exhibits only early signs of a disease, wherein treatment is administered for the purpose of decreasing the risk of developing a pathology associated with the disease. In one embodiment, the compounds of the invention may be given as a prophylactic treatment to prevent undesirable or unwanted weight gain.

A “therapeutic treatment” is a treatment administered to a subject who exhibits signs or symptoms of pathology, wherein treatment is administered for the purpose of diminishing or eliminating those pathological signs.

“Diseases or conditions responsive to administration of a modulator (e.g. inhibitor) of ceramidase activity include obesity, overweight, appetite disorder, a metabolic disorder, cellulite, Type I and Type II diabetes, hyperglycemia, dyslipidemia, Syndrome X, insulin resistance, diabetic dyslipidemia, hyperlipidemia, bulimia, hyperlipidemia, hypercholesterolemia, hypertriglyceridemia, artherogenesis, artherosclerosis, Alzheimer disease, an inflammatory disorder, vascular inflammation, inflammatory bowel disorder, Crohn's disease, rheumatoid arthritis, asthma, thrombosis or cachexia.

The term “to control weight” includes the loss of body mass or the reduction of weight gain over time.

Disorders associated with impaired appetite regulation, that may be treated by the compounds of the invention, are understood to include disorders associated with the intake of one or more substance, especially the abuse and/or dependency on a substance, or a disorder of food behaviour in particular behaviour liable to cause excess weight such as bulimia, appetency for sugars, non-insulin-dependent diabetes. Said one or more substance includes foods and their ingredients, such as sugars, carbohydrates, fats as well as drinking alcohol, drugs of abuse or addiction, or excessive consumption. An impaired appetite regulation may be associated with an “appetite” directed to said substances, and the uncontrolled, or dependent, or excessive consumption of said substances.

Compounds of the invention with the chemical structure of formula I and II (for example; ceramidase inhibitors), OEA-like compounds, OEA-like modulators, may possess asymmetric carbon atoms (optical centers) or double bonds; and encompass racemates, diastereomers, geometric isomers and individual isomers of said compounds.

The compounds of the invention may be separated into diastereoisomeric pairs of enantiomers by fractional crystallization from a suitable solvent, for example methanol or ethyl acetate or a mixture thereof. The pair of enantiomers thus obtained may be separated into individual stereoisomers by conventional means, for example by use of an optically active acid as a resolving agent. Alternatively, any enantiomer of such compound of the invention may be obtained by stereospecific synthesis using optically pure starting materials of known configuration.

The compounds of the invention may furthermore have unnatural ratios of atomic isotopes at one or more of their atoms, and may for example by radiolabeled with isotopes, such a tritium or carbon-14.

The compounds of the invention may be isolated in the form of their pharmaceutically acceptable acid addition salts, such as the salts derived from using inorganic and organic acids. Suitable acids include hydrochloric, nitric, sulphuric, phosphoric, formic, acetic, trifluoroacetic, propionic, maleic, succinic, and malonic. In one embodiment compounds of the invention containing an acidic function can be in the form of their inorganic salt in which the counterion can be selected from sodium, potassium, lithium, calcium, magnesium, and organic bases. The term “pharmaceutically acceptable salts” means salts prepared from pharmaceutically acceptable non-toxic bases or acids including inorganic bases or acids and organic bases or acids.

The compounds of the invention further encompass prodrugs of compounds with the chemical structure formula I or II (for example, ceramidase inhibitors), OEA-like compounds and OEA-like modulators, which on administration undergo chemical conversion by metabolic processes before becoming active pharmacological substances. Prodrugs include derivatives of the compounds of the invention that are readily convertible in vivo into a functional compound of the invention. Procedures suitable for the preparation of said prodrug derivatives are described in “Design of Prodrugs” ed. H. Bundgaard, Elsevier, 1985.

I. A Compound that is an Appetite Suppressing Agent

A compound that is an appetite suppressing regulation or satiety inducing agent, such as ceramidase inhibitor, homologue, or analogue of the present invention is a synthetic ceramide based on hydrophobic phenylalcohols having the structure:

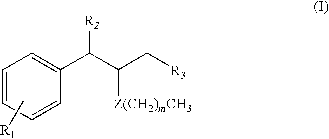

In this formula, m is an integer ranging from 0 to 22. Z is a member selected from —C(O)N(R₄)—; —(R₄)NC(O)—; —OC(O)—; —(O)CO—; O; NR₄; and S, in which R₁, R₂, R₃, and R₄ are independently selected from the group consisting of substituted or unsubstituted alkyl, hydrogen, NO₂, OH, methoxy, chlorine, bromine, fluorine, substituted or unsubstituted C₁-C₆ alkyl, substituted or unsubstituted lower (C₁-C₆) acyl, ether, homoalkyl, and aryl. Up to twelve hydrogen atoms of the compound may also be substituted by a methyl group, a double bond, or a triple bond.

Another example of a chemical compound of the invention that is an appetite suppressing regulation or satiety inducing agent is a synthetic ceramide compound that also includes an N-acyl-phenylaminoalcohol analog of the following formula:

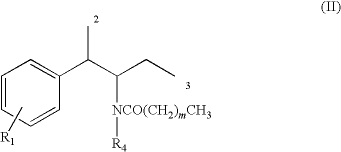

In one embodiment, the compounds of Formula II have m from 6 to 18; and members R₁, R₂, R₃, and R₄ are independently selected from the group consisting of substituted or unsubstituted alkyl, hydrogen, NO₂, OH, methoxy, chlorine, bromine, fluorine, substituted or unsubstituted C₁-C₆ alkyl, substituted or unsubstituted lower (C₁-C₆) acyl, ether, homoalkyl, and aryl. In this embodiment up to twelve hydrogen atoms of the fatty acid portion and phenylaminoalcohol (e.g. phenylaminopropanol) portion of compounds of the above formula may also be substituted by a methyl or a double bond. In some embodiments, up to twelve hydrogen atoms of the fatty acid portion of Formula II may be substituted by a methyl, a double bond, or a triple bond. In some embodiments with acyl groups, the acyl groups may be the propionic, acetic, or butyric acids and attached via an ester linkage as R₁, R₂, and R₃ or an amide linkage as R₄. In some embodiments, a hydrogen atom attached to a carbon atom of a compound of the above formula is replaced with a halogen atom, a chlorine atom or a fluorine atom.

In another embodiment, the above compounds particularly include those in which the fatty acid moiety comprises lauric acid, myristic acid, or palmitic acid. Such compounds include N-lauroyl-2-amino-1-phenyl-1-propanol, N-lauroyl-2-amino-1-(4′-nitrophenyl)-1,3-propandiol, N-myristoyl-2-amino-1-phenyl-1-propanol, N-myristoyl-2-amino-1-(4′-nitrophenyl)-1,3-propandiol, N-palmitoyl-2-amino-1-phenyl-1-propanol, and N-palmitoyl-2-amino-1-(4′-nitrophenyl)-1,3-propandiol.

In still another embodiment, the compounds of Formula II have m from 10 to 14; and members R₁, R₂, R₃, and R₄ are independently selected from the group consisting of substituted or unsubstituted alkyl, hydrogen, NO₂, OH, methoxy, chlorine, bromine, fluorine. In this embodiment up to six hydrogen atoms of the fatty acid portion and phenylaminoalcohol (e.g. phenylaminopropanol) portion of compounds of the above formula may also be substituted by a methyl or a double bond. In some embodiments, up to six hydrogen atoms of the fatty acid portion of Formula II may be substituted by a methyl, a double bond, or a triple bond.

In one embodiment the compounds of Formula II have m from 10 to 14. In other embodiments of the invention, m is 10 or 12. In this embodiment R₁ is for example H, NO₂, OH, chlorine, bromine, or fluorine. R₁ can be situated at any position on the phenyl ring of Formula II, for example at the 4′ position. R₂ is for example hydrogen, OH, or methoxy. R₃ is such as H or OH. R₄ is for example hydrogen.

Exemplary compounds provide hydroxy, methoxy, and NO₂ substituted compounds, including N-acyl-2-amino-1-phenyl-1-propanol (N-acyl-AAP), N-acyl-2-amino-1-(4′-nitrophenyl)-1,3-propandiol (N-acyl-AAPD), of Formula II.

Such compounds include:

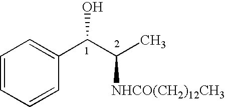

(1S,2R) D-erythro-N-myristoyl-2-amino-1-phenyl-propanol [D-erythro-MAPP]

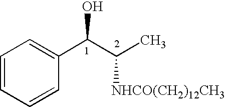

(1R,2S) L-erythro-N-myristoyl-2-amino-1-phenyl-propanol [L-erythro-MAPP]

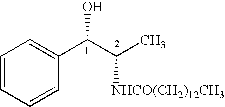

(1S,2S) L-threo-N-myristoyl-2-amino-1-phenyl-propanol [L-threo-MAPP]

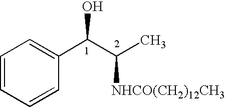

(1R,2R) D-threo-N-myristoyl-2-amino-1-phenyl-propanol [D-threo-MAPP]

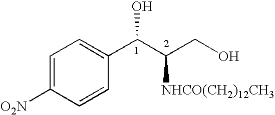

(1S,2R) D-erythro-N-myristoyl-2-amino-1-(4′-nitrophenyl)-1,3-propandiol [D-erythro-NMAPPD]

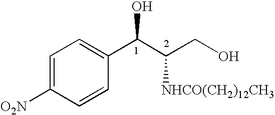

(1R,2S) L-erythro-N-myristoyl-2-amino-1-(4′-nitrophenyl)-1,3-propandiol [L-erythro-NMAPPD]

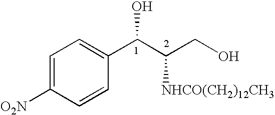

(1S,2S) L-threo-N-myristoyl-2-amino-1-(4′-nitrophenyl)-1,3-propandiol [L-threo-NMAPPD]

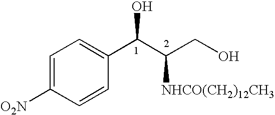

(1R,2R) D-threo-N-myristoyl-2-amino-1-(4′-nitrophenyl)-1,3-propandiol [D-threo-NMAPPD]
said N-acyl-phenylaminoalcohol is (1S,2R) D-erythro-N-myristoyl-2-amino-1-phenyl-propanol [D-erythro-MAPP] or (1R,2R) D-threo-N-myristoyl-2-amino-1-(4′-nitrophenyl)-1,3-propandiol [D-threo-NMAPPD].

In one embodiment the invention provides a compound with the chemical structure of formula I or II that is an inhibitor that is functionally characterised by its ability to inhibit the hydrolytic activity of a member of the enzymatic class of hydrolytic enzymes (hydrolases) acting on carbon-amide bonds, other than peptide bonds, in linear amides, more specifically enzymes that can be characterized as a ceramidase. For examples theceramidase, which is inhibited, regulates the level of anorexic lipids (e.g. oleoylethanolamide, palmitoylethanolamide, elaidoylethanolamide or oleoyl-estrone) in a mammal, in particular a ceramidase (e.g. neutral ceramidase) that is expressed in the intestine. The effect of a compound of the invention on ceramidase activity is tested in a standard (in vitro) assay e.g. as described by Bielawska et al. (17).

For example, the ceramidase inhibitor of the invention, having the structures (1S,2R) D-erythro-N-myristoyl-2-amino-1-phenyl-propanol [D-erythro-MAPP] and (1R,2R) D-threo-N-myristoyl-2-amino-1-(4′-nitrophenyl)-1,3-propandiol are commercially available from e.g. Cayman Chemical Company (Ann Arbor, Mich., USA). Alternatively the above-mentioned compounds may be synthesized as described in detail by Bielawska et al. (17).

The compound of this invention, such as a ceramidase inhibitor, homologue, or analogue thereof, is formulated as a pharmaceutical or cosmetic preparation or dietary supplement for administration either alone, or in combination with an anorexic acylamide e.g. oleoylethanolamide, (or palmitoylethanolamide, elaidoylethanolamide) or oleoyl-estrone.

### II. Further Appetite Suppressant Compounds and Analogues of the Invention

A further appetite suppressant compound of the invention is an “N-acylamine-containing compound” comprising the moiety R—C(═O)—NH— and having the structure [formula III]:

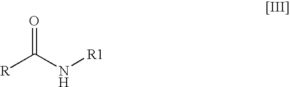

According to the invention, R—C═O is an acyl derivative of a natural or synthetic fatty acid, where the hydroxyl-group has been removed from the carboxylic acid group of the fatty acid.

Said fatty acid may be a branched or unbranched, cyclic or acyclic, substituted or unsubstituted chain of from 3 to 28 carbon atoms, such as, e.g. from 14 to 22 carbon atoms. Said fatty acid may be saturated, i.e. contains no double- or triple bonds; or it may be unsaturated and contain from 1 and 6 double bonds, such as, e.g., from 1 to 3 double bonds. Furthermore, said fatty acid may contain from 1 to 4 triple bonds, 1 or 2 triple bonds.

Table I and Table II give examples of fatty acids, that are suitable for use as the R—C═O component.

TABLE I
Fatty acids that are suitable for use as the R—C═O component

| | Trivial | |
|---|---|---|
| Carbon Number | Nomenclature | IUPAC Nomenclature |
| 3:0 | Propinic acid | Trianoic acid |
| 4:0 | Butyric acid | Tetranoic acid |
| 5:0 | Valeric acid | Pentanoic acid |
| 6:0 | Capric acid | Hexanoic acid |
| 7:0 | Heptanic acid | Heptanoic acid |
| 8:0 | Caprylic acid | Octanoic acid |
| 9:0 | Nonanic acid | Nonanoic acid |
| 10:0 | Capryl | Decanoic acid |
| 11:0 | Undecanic acid | Undecanoic acid |
| 12:0 | Lauric acid | Dodecanoic acid |
| 13:0 | Tridecanic acid | Tridecanoic acid |
| 14:0 | Myristic acid | Tetradecanoic acid |
| 14:1 | Myristoleic acid | 9-cis-Tetradecenoic acid |
| 14:1 | Myristelaidic acid | 9-trans-Tetradecenoic acid |
| 15:0 | Pentadecanic acid | Pentadecanoic acid |
| 16:0 | Palmitic acid | Hexadecanoic acid |
| 16:0 [(CH₃)₄] | Phytanic acid | 3,7,11,15-Tetramethylhexadecanoic acid |
| 16:1 | Palmitoleic acid | 9-cis-Hexadecenoic acid |
| 16:1 | Palmitelaidic acid | 9-trans-Hexadecenoic acid |
| 17:0 | Heptadecanic acid | Heptadecanoic acid |
| 18:0 | Stearic acid | Octadecanoic acid |
| 18:1 | Petroselinic acid | 6-cis-Octadecenoic acid |
| 18:1 | Oleic acid | 9-cis-Octadecenoic acid |
| 18:1 | Elaidic acid | 9-trans-Octadecenoic acid |
| 18:1 | Ricinoleic acid | 12-Hydroxy-9-cis-octadecenoic acid |
| 18:1 | Ricinelaidic acid | 12-Hydroxy-9-trans-octadecenoic acid |
| 18:1 | Vaccenic acid | 11-cis-Octadecenoic acid |
| 18:1 | Trans-Vaccenic acid | 11-trans-Octadecenoic acid |
| 18:2 | Linoleic acid | 9-cis-12-cis-Octadecadienoic acid |
| 18:2 | Conjugated linoleic acids | 9-cis-11-trans-Octadecadienoic acid and |
| | | 10-trans-12-cis-octadecadienoic acid |
| 18:2 | Linoelaidic acid | 9-trans-12-trans-Octadecadienoic acid |
| 18:3 | Linolenic acid | 9-cis-12-cis-15-cis-Octadecatrienoic acid |
| 18:3 | γ-Linolenic acid | 6-cis-9-cis-12-cis-Octadecatrienoic acid |
| 18:3 | Conjugated linolenic acids | 6-cis-9-cis-11-trans-octadecatrienoic acid and |
| | | 8-cis-11-cis-13-trans-Octadecatrienoic acid |
| 19:0 | Nonadecanic acid | Nonadecanoic acid |
| 20:0 | Arachidic acid | Eicosanoic acid |
| 20:1 | Eicosenic acid | 11-cis-Eicosenoic acid |
| 20:3 | Homo-γ-linolenic acid | 8-cis-11-cis-14-cis-Eicosatrienoic acid |
| 20:4 | Arachidonic acid | 5,8,11,14 (all cis) Eicosatetraenoic acid |
| 20:5 | — | 5,8,11,14,17 (all cis) Eicosapentaenoic acid |
| 21:0 | Heneicosanic acid | Heneicosanoic acid |
| 22:0 | Behenic acid | Docosanoic acid |
| 22:1 | Erucic acid | 13-cis-Docosenoic acid |
| 22:6 | — | 4,7,10,13,16,19 (all cis) Docosahexaenoic acid |
| 23:0 | Tricosanic acid | Tricosanoic acid |
| 24:0 | Lignoceric acid | Tetracosanoic acid |
| 24:1 | Nervonic acid | 15-cis-Tetracosenoic acid |
| 26:0 | Cerotic acid | Hexacosanoic acid |

TABLE II
Fatty acids (18) that are suitable for use as the R—C═O component
Fatty acid

Undeca-2E,4Z-diene-8,10-diynoic acid
Undeca-2E,4E-diene-8,10-diynoic acid
Dodeca-2E,4Z-diene-8,10-diynoic acid
Dodeca-2E,4E,10E-trien-8-ynoic acid
Trideca-2E,7Z-diene-10,12-diynoic acid
Dodeca-2E,4E,8Z,10E-tetraenoic acid
Dodeca-2E,4E,8Z,10Z-tetraenoic acid
Dodeca-2E,4E,8Z-trienoic acid
Dodeca-2E,4E-dienoic acid
Undeca-2E-ene-8,10-diynoic acid
Undeca-2Z-ene-8,10-diynoic acid
Dodeca-2E-ene-8,10-diynoic acid
Dodeca-2E,4Z,10Z-trien-8-ynoic acid
Pentadeca-2E,9Z-diene-12,14-diynoic acid
Hexadeca-2E,9Z-diene-12,14-diynoic acid

The synthetic fatty acids are fatty acids wherein one or more carbon atoms have been replaced by other atoms, such as, e.g., sulphur atoms.

In one embodiment of the invention, R1 is i) a branched or unbranched, saturated or unsaturated, substituted or unsubstituted chain of from 1 to 30 carbon atoms, which optionally is substituted with one or more hydroxy groups, which may be primary, secondary or tertiary, or ii) an N-terminal amino acid or peptide residue.

In an alternative embodiment of the invention R1 is an alk amine optionally substituted by one or more hydroxy groups, wherein alk is alkyl or alkenyl. Where said alk amines are without any hydroxy groups, they are isobutylamine or 2-methylbutylamine. Alternatively where said alk amines are substituted with hydroxy groups, they may be alkanolamines, such as ethanolamine or propan-1-ol-2-amine. Furthermore said alk amine may alternatively be an amino acid residue or a peptide.

In a further embodiment of the invention the R1 group is a sphingoid base, such as sphingosin or sphinganin.

Accordingly, the anorexic acylamide compounds of the invention having R- and R1-groups according to the above definitions may be an N-acylalkanolamine, such as an N-acylethanolamine. Said N-acylethanolamines compounds, may be N-oleoylethanolamine, N-palmitoylethanolamine, N-linoleoylethanolamine, N-α-linolenoylethanolamine or N-γ-linolenoylethanolamine.

In an alternative embodiment, said anorexic acylamide compounds of the invention may be an N-acylpropan-1-ol-2-amine, such as N-oleoylpropan-1-ol-2-amine or N-arachidonoylpropan-1-ol-2-amine. The N-acylamine containing compounds according to the invention may also be composed of acyl derivatives of the fatty acids mentioned in Table II and isobutylamine or 2-methylbutylamine.

In another embodiment of the invention the compound may be a N-acylpropan-1-ol-2-amine, such as N-oleoylpropan-1-ol-2-amine or N-arachidonoylpropan-1-ol-2-amine.

The N-acylamine containing compounds according to the invention may also be composed of acyl derivatives of the fatty acids mentioned in Table II and isobutylamine or 2-methylbutylamine.

An anorexic acylamide compound of the invention is functionally characterised by its ability to act as a potent body fat and weight controlling compound that acts by suppressing appetite and/or enhancing satiety, and reducing energy intake.

The anorexic acylamide of the invention, having the structure N-acylethanolamide including oleoylethanolamide is commercially available from e.g. Sigma-Aldrich (St. Louis, Mo., USA). Alternatively, said N-acylethanolamide may be synthesized as described in detail by Abadji et al. (19).

III Use of a Compound of Formular I or II, Either Alone, or in Combination with an Anorexic Acylamide, for the Manufacture of a Preparation for use in Prophylactic or Therapeutic Treatment, or Cosmetic Treatment, or as a Dietary Supplement, to Reduce Energy Intake in a Mammal

The invention relates to a method of modifying the feeding behaviour of a mammal e.g. human and/or a domestic animal. The method comprising administering to a mammal such as, e.g. a human and/or a domestic animal in need thereof, a composition comprising an effective amount of a compound according to formula I or formula II, such as a ceramidase inhibitor, homologue, or analogue thereof, either alone, or in combination with an effective amount of an anorexic acylamide e.g. oleoylethanolamide, (or palmitoylethanolamide, elaidoylethanolamide) or oleoyl-estrone. Said composition may be formulated for administration as a pharmaceutical or cosmetic preparation, or as a dietary supplement.

According to the method of the present invention said modified feeding behaviour of said mammal may comprise a suppression of hunger, and/or an enhancement of satiety, and may be accompanied by a reduction in energy intake of a mammal. Furthermore the method of the invention may be employed to reduce the fat tissue mass/lean mass ratio in a human or domestic animal.

In a further embodiment, the method may be employed as a cosmetic treatment to reduce body weight in a mammal, in particular a human or a domestic animal in need thereof.

According to the present invention an “Overweight” human is a human having a BMI in a range from about 25 to about 29.9, wherein the term “body mass index” or “BMI” is defined as body weight (kg)/height² (m²). Furthermore, “Obesity” in a human is intended to indicate a human having a BMI, which is at least about 30.

A composition that provides an effective amount of a compound according to formula I or formula II, such as a ceramidase inhibitor, homologue, or analogue thereof, of the invention, either alone, or in combination with an effective amount of an anorexic acylamide e.g. oleoylethanolamide, (or palmitoylethanolamide, elaidoylethanolamide) or oleoyl-estrone is one that may be used in preventing and treating obesity and the accompanying diseases (e.g. type 2 diabetes and other obesity associated diseases, such as coronary heart disease) in humans; as well as for modifying the feeding behaviour of a mammal, for suppression of hunger, enhancement of satiety or reducing energy intake of a mammal, for reducing the fat tissue mass/lean mass body mass ratio and for a cosmetic method for reducing body weight. The composition, wherein the weight ratio of said ceramidase inhibitor and said anorexic acylamide ranges from about 1:10,000 to 10,000:1, may be administered in a total amount of about 0.1 μg/kg to about 2 g/kg body weight, such as, e.g., from about 750 mg/kg to about 2 g/kg body weight, from about 1 μg/kg to 750 mg/kg body weight, from about 10 μg/kg to about 500 mg/kg body weight, from about 0.1 mg/kg to about 250 mg/kg, from about 1 mg/kg to about 100 mg/kg body weight or from about 10 mg/kg to about 50 mg/kg body weight.

In a further embodiment the administered composition may comprise a combination of two or more of said compounds according to formula I or formula II, such as ceramidase inhibitors or two or more of said anorexic acylamide compounds.

IV Formulation of a Composition Comprising Compound Having Formular I or II Alone, or in Combination with an Anorexic Acylamide for use as a Medicament, Cosmetic Preparation, or as a Dietary Supplement.

A composition comprising an effective amount of a compound according to formula I or formula II, such as a ceramidase inhibitor, homologue, or analogue thereof, of the invention, either alone, or in combination with an effective amount of an anorexic acylamide e.g. oleoylethanolamide, (or palmitoylethanolamide, elaidoylethanolamide) or oleoyl-estrone, may be formulated alone or together with a pharmaceutically acceptable excipient.

A composition according to the invention may be formulated for administration by any means known in the art, including compositions suitable for oral, rectal, topical, subdermal, parenteral (including subcutaneous, intraperitoneal, intramuscular, and intravenous), ocular (ophthalmic), pulmonary (nasal or buccal inhalation), or nasal administration, although the most suitable route in any given case well depend in part on the nature and severity of the conditions being treated and on the nature of the active ingredient. Exemplary routes of administration are oral and intraperitoneal. The compositions may be conveniently presented in unit dosage form and prepared by any of the methods well-known in the art of pharmacy.

The formulated composition according to the invention may be administered as often as required to effect a reduction in energy intake, suppression of hunger, increase in satiety, for example hourly, every six, eight, twelve or eighteen hours, daily or weekly.

Formulations suitable for oral administration include (a) liquid solutions; (b) tablets, capsules, sachets, each containing a predetermined amount of the one or more active ingredients of the composition in the form of liquids, solids (e.g. powders, granules); (c) suspensions in a liquid excipient, (d) emulsions.

Formulation in tablet form may include one or more pharmaceutical excipients, i.e. a therapeutically inert substance or carrier such as lactose, sucrose, mannitol, sorbitol, calcium phosphates, corn starch, potato starch, microcrystalline starch, gelatin, colloidal silicon dioxide, talc, magnesium stearate, stearic acid and other excipients, colorants, fillers, binders, disintegrating agents, diluents, glidants, solvents, emulsifying agents, suspending agents, stabilizers, enhancers, pH adjusting agents, retarding agents, wetting agents, surface active agents, preservatives. Where a formulation is in lozenge form it may include a flavour e.g. sucrose, while in pastille form it may include an inert base e.g. gelatin, glycerin, or sucrose and acacia emulsions, or gels. The dosage form may be designed to release the compound freely or in a controlled manner e.g. with respect to tablets by suitable coatings.

Formulations comprising the composition of the invention that are suitable for injection may include aqueous or non-aqueous, isotonic sterile injection solutions, which may contain antioxidants, buffers, bacteriostats.

The composition of the invention in any of the contemplated pharmaceuticals may comprise from about 0.1 to about 100% w/w of the pharmaceutical composition, and prepared by any of the methods well known to a person skilled in the art. Details can be found in pharmaceutical handbooks such as, e.g., Remington's Pharmaceutical Sciences (Mack Publ Co. Eston) or Pharmaceutical Excipient Handbook.

The composition of the invention may also be provided in the form of a dietary supplement e.g. a herbal composition for oral administration, comprising a herbal extract from Echinacea, peas, oats, potatoes, cotton, tobacco, wheat, rice, soy, peanuts, corn or tomatoes, wherein a compound according to the invention is present.

V Use of a Compound According to Formula I or Formula II, Such as a Ceramidase Inhibitor in Combination with a Weight Control Drug, for the Manufacture of a Preparation for Use in Therapeutic Treatment, or Cosmetic Treatment, or as a Dietary Supplement, to Reduce Energy Intake in a Mammal

A compound according to formula I or formula II of the invention, for example ceramidase inhibitor, may alternatively be combined with an active pharmaceutical ingredient amenable for treatment of metabolic disorders, wherein said ingredient is selected from the following group: compounds that function as centrally-acting releasers of endogenous monoamines, noradrenalin and dopamine (for example phentermine); compounds that are pancreas lipase inhibitors (for example orlistat); compounds that are centrally-acting inhibitors of re-uptake of the monoamines, noradrenaline and serotonin (for example sibutramine); compounds that are antagonists of cannabinoid receptors type 1 (for example rimonabant); and compounds that are agonists of PPARα (for example fenofibrate and β-fibrate).

### EXAMPLES

The following experimental details relate to the examples that follow:

Mice and rats are housed in cages in a temperature- and light-controlled stable with 12 h light/dark cycle (lights on at 03:00 AM and lights off at 03:00 PM). In order to avoid hydrolysis of OEA by the enzyme fatty acid amide hydrolase (FAAH), FAAH knock out mice are used. Alternatively, 0.01-10 mg/kg of the specific FAAH inhibitor URB597 (cyclohexyl carbamic acid 3′-carbamoyl-biphenyl-3-yl ester) (14) is either systemically administered to a group of wild type mice or rats ½-72 h before euthanization and subsequent removal of the intestines, which is a well-documented method for specific inhibition of FAAH (14), or URB597 is added directly to the in vitro assay in concentrations of 1-10 μM, which has previously been shown to inhibit FAAH activity completely in a standard in vitro FAAH assay at pH 9 utilizing 100 μM of anandamide (AEA) as substrate and 14 μg/μl of rat liver membrane preparation as protein source (20).

Collection of intestinal protein is carried out on rodents fasted overnight and anaesthetized (i.m.) with e.g. Ketalar-Rompun (2:1) (Ketalar, 50 mg/ml, Parke Davis, Detroit, Mich.: Rompun Vet, 20 mg/ml). A segment of small intestine from 5-10 cm below the pylorus to 5-10 cm above the cecum is cannulated. The segment is flushed twice with 20 ml 0.9% NaCl containing 1 mM benzamidine, 1 mM PMSF, and 3 mM taurodeoxycholate, which serves to dissociate ceramidase from the intestinal brush border. The eluted solution is centrifuged at 3,000×g at 0° C., and the supernatant is concentrated by ultrafiltration through a YM-30 membrane (Millipore, Billerica, Mass., USA) according to (16). The concentrated supernatants are used as intestinal protein. Alternatively, intestinal tissue (e.g. jejunum) from rats is homogenized and centrifuged at 1,000×g for 10 min. The supernatant is then used as a source of intestinal protein.

Protein determination is performed by the method of Bradford using γ-globulin or bovine serum albumin as a protein standard. All below data is obtained within the linear range of protein added and time of incubation.

Enzymatic hydrolysis of OEA is investigated in an assay containing 5-500 μg of intestinal protein incubated with 1-500 μM of ³H-OEA ([1-³H-ethanolamine]OEA and dilutions thereof with non-radioactive OEA to obtain a specific activity of 0.1-100,000 dpm/pmol) or ³H-anandamide ([1−³H-ethanolamine]anandamide and dilutions thereof with non-radioactive anandamide to obtain a specific activity of 0.1-100,000 dpm/pmol) in a total volume of 100-500 μl of 10-100 mM buffer (pH 3-11), initially utilizing 100 mM Tris-HCl (pH 8.0) containing 0-2 mM EDTA and 0-5 mg/ml of fatty acid-free bovine serum albumin with or without addition of D-erythro-MAPP (or a related compound; dissolved in 1-10 μl ethanol) in a final concentration of 0.0001-1000 μM (1-10 μl ethanol is added to control assays). Incubations are carried out at 37° C. for 0-120 min and samples are withdrawn at 2-3 time points. The reaction in a sample is terminated by addition of 200-1000 μl of chloroform:methanol (1:1 v/v) and cooling on ice, followed by 10 min centrifugation at low speed, where after 50-250 μl of the upper phase is extracted and the radio-labeled product ethanolamine formed is quantified by liquid scintillation counting.

Inhibition of FAAH enzymatic activity in intestinal tissue of URB597-treated mice and rats, is tested employing the above described assay for enzymatic hydrolysis of OEA, using anandamide as substrate instead of OEA.

Enzymatic hydrolysis of ceramide is measured in an assay containing 5-500 μg of intestinal protein incubated with 1-5000 μM of ¹⁴C-ceramide [(N-[1-¹⁴C]acyl-D-erythro sphingosine (with the acyl group being from six to twenty carbon atoms) and dilutions thereof with non-radioactive ceramide to obtain a specific activity of 0.1-100,000 dpm/pmol], which is sonicated in buffer or added in 5-50 μl of ethanol, to a total volume of 50-500 μl of 10-100 mM buffer (pH 3-11) initially utilizing 100 mM Tris-HCl (pH 8.0) containing 0-100 mM CHAPS, 0-10 mM DTT, 0-1% Nonidet P-40, 0-5 mg/ml of bovine serum albumin, and 0-500 mM NaCl with or without addition of D-erythro-MAPP (or a related compound; dissolved 1-10 μl ethanol) in a final concentration of 0.0001-1000 μM (1-10 μl ethanol is added to control assays). Incubations are carried out at 37° C. for 0-120 min and samples are withdrawn at 2-3 time points. The reaction in each sample is terminated by addition of 200-1000 μl of methanol:chloroform:heptane (28:25:20 v/v/v) and 50-400 μl of potassium carbonate buffer (pH 10). After mixing, the samples are centrifuged 1-20 min at low speed centrifugation and 50-400 μl of the upper phase is extracted and the radio-labeled product, free fatty acid, in the upper phase is quantified by liquid scintillation counting. From selected samples, another 50-400 μl aliquot of the upper phase as well as 50-400 μl of the lower phase are evaporated to dryness under a stream of nitrogen, re-dissolved in 10-200 μl of chloroform:methanol (19:1 v/v) and applied on thin-layer chromatography plates, eluted in chloroform:methanol:acetic acid (94:1:5 v/v/v) followed by quantification of the distribution of the radiolabeled free fatty acid in the two phases using a Phosphorlmager scanner (STORM; Molecular Dynamics, Sunnyvale, Calif., USA) and ImageQuant software (GE Health Care, Amersham, United Kingdom). The distribution of released fatty acid is used to calculate the total amount of product formed in each sample.

OEA and ceramide hydrolysis assays are carried as described above, further varying the pH conditions, where the buffer composition is varied to be within the buffer zone of each buffer used: In the range pH 3-7 10-200 mM citrate-NaHPO₄ is used, in the range pH 7-9 10-200 mM Tris-HCl is applied, alternatively in the range pH 7-10 10-200 mM bis-tris propane is applied, and in the range pH 9-11 10-200 mM Na₂CO₃—NaHCO₃ or 10-200 mM glycin-NaOH is utilized.

Food intake studies are performed with male Sprague-Dawley rats (250-350 g) or male C57BL/6J mice (Charles River, Sulzfeld, Germany). The animals are acclimatized for two weeks and subsequently transferred to individual cages with ad libitum access to tap water and powdered chow via the mounted feeders (e.g. MANI FeedWin system) or offered an energy-dense high-fat diet (60% energy from fat; Research Diets, New Jersey, USA; due to the high fat content and hence susceptibility to harshness new food is offered every other day and the old food is discarded). These animals are left on the diet for 12 weeks before the experiment is commenced. The animals are group randomized into weight-matched groups of n=4-12 to receive up to four administrations (0.1-10 ml/kg p.o., i.p., s.c., or i.v.) of a test compound (e.g. MAPP or NMAPPD) or vehicle (saline optionally with Tween 80, polyethylene glycol, DMSO, ethanol, Cremophor excipient) with at least seven days interval. All compounds are administered just prior to the beginning of the dark period optionally after a 24 h fasting period. Food intake (digital balance) and water intake (registered gravimetrically or by means of lick counts) and activity (consecutive beam breaks) are monitored for at least 12 hours following the time of injection. After the treatment, the animals are euthanized by use of CO₂ followed by decapitation.

### Example 1

### Effects of D-erythro-MAPP, D-threo-NMAPPD, and URB597 on A) OEA and Anandamide Hydrolysis and B) Ceramide Hydrolysis by Intestinal Protein from (URB597 Treated) Rats

The aim of the in vitro studies (examples 1-2) is to prove that part of OEA hydrolysis in intestinal tissue is due to an enzyme different from FAAH, which is characterized as an acylethanolamide/acylamide hydrolyzing enzyme with approximately 5-fold preference for anandamide (AEA) compared to OEA (21).

1A: OEA hydrolysis is determined in an assay modified from Fegley et al. (14) with 50 μg of intestinal protein from male Sprague-Dawley rats (approximately 200 g), URB597 (Cayman Chemical, Ann Arbor, Mich., USA) treated (0.3 mg/kg i.p. 1 h prior to anaesthesia) and untreated [4 ml/kg of vehicle (saline/Tween 80/polyethylene glycol; 90:5:5) i.p. 1 h prior to anaesthesia], incubated with 28 μM of [1−³H-ethanolamine]OEA (from American Radiolabeled Chemicals, Inc., St. Louis, Mo., USA diluted with non-labeled OEA from Sigma Aldrich, St. Louis, Mo., USA to a specific activity of 10 dpm/pmol) in duplicate for 0, 10, 20, and 30 min at 37° C. in a total volume of 200 μl of 100 mM Tris-HCl buffer (pH 8.0) containing 0.9 mM EDTA, 1.5 mg/ml of fatty acid-free bovine serum albumin, and 5 μl ethanol with 0; 0.04; 0.4; 4; 40; 400 μM D-erythro-MAPP or D-threo-NMAPPD (Cayman Chemical, Ann Arbor, Mich., USA) giving a final concentration of D-erythro-MAPP or D-threo-NMAPPD of 0; 0.001; 0.01; 0.1; 1; and 10 μM. The reaction in a sample is terminated by addition of 400 μl chloroform:methanol (1:1 v/v) and cooling on ice, followed by 10 min centrifugation at low speed, where after 100 μl of the upper phase is extracted and the radio-labeled product ethanolamine formed is quantified by liquid scintillation counting. In another set of experiments the following assay conditions are adjusted—in order to be identical to those of the ceramidase assay (see example 1B)—by using 25 μg of protein from rat intestinal (jejunum) homogenate in a total volume of 100 μl, 50 μM of ³H-oleoylethanolamide (OEA; 25.000 dpm) or ³H-anandamide (AEA; 25.000 dpm), 100 mM citrate phosphate (7.0), 8 mM CHAPS. 0; 2; 20; and 200 μM of URB597 (Cayman Chemical, Ann Arbor, Mich., USA) was added in 5 μl of DMSO giving a final concentration of URB597 of 0; 0.1; 1; and 10 μM while the ceramidase inhibitors, D-erythro-MAPP and D-threo-NMAPPD are added in concentrations of 0; 1; 2; 10; and 20 mM in 5 μl of ethanol resulting in final concentrations of 0; 50; 100; 500; and 1000 μM. Incubation at 37° C. is carried out for 0 and 20 min after establishment of linearity of product formation within this time frame.

1B: Ceramide hydrolysis is determined in an assay according to (16) with 50 μg of intestinal protein from male Sprague-Dawley rats (approximately 200 g), URB597 treated (0.3 mg/kg i.p. 1 h prior to anaesthesia) and untreated [4 ml/kg of vehicle (saline/Tween 80/polyethylene glycol; 90:5:5) i.p. 1 h prior to anaesthesia], incubated for 0, 15, 30, and 60 min with 0.5 mM of N-[1-¹⁴C]oleoyl-D-erythro sphingosine diluted with non-labeled N-oleoyl-D-erythro sphingosine (both from American Radiolabeled Chemicals, Inc., St. Louis, Mo., USA) in duplicates to a specific activity of 500 dpm/nmol and added in 5 μl of ethanol to a total volume of 100 μl 100 mM Tris-HCl buffer (pH 8.0) containing 8 mM CHAPS and 2.5 μl ethanol with 0; 0.04; 0.4; 4; 40; 400 μM D-erythro-MAPP (Cayman Chemical, Ann Arbor, Mich., USA) giving a final concentration of D-erythro-MAPP of 0; 0.001; 0.01; 0.1; 1; and 10 μM. The reaction in a sample is terminated by addition of 600 μl methanol:chloroform:heptane (28:25:20 v/v/v) and 200 μl 0.05 M potassium carbonate buffer (pH 10). The potassium carbonate buffer used in the equilibrations is a potassium carbonate-potassium borate-potassium hydroxide buffer, 0.05 M (Standard Buffer Solution, Fischer Scientific UK Limited, Loughborough, UK). After mixing, the samples are centrifuged for 10 min at low speed, and 200 μl of the upper phase is extracted and the radio-labeled product, free fatty acid, in the upper phase is quantified by liquid scintillation counting. In another set of experiments the following assay conditions are adjusted—in order to be identical to those of the FAAH assay (see example 1A)—by using 25 μg of protein from rat intestinal (jejunum) homogenate in a total volume of 100 μl, 50 μM of ¹⁴C-Octanoyl-D-sphingosine (ceramide; 25.000 dpm), 100 mM citrate phosphate (7.0), 8 mM CHAPS. 0; 2; 20; and 200 μM of URB597 (Cayman Chemical, Ann Arbor, Mich., USA) was added in 5 μl of DMSO giving a final concentration of URB597 of 0; 0.1; 1; and 10 μM while the ceramidase inhibitors, D-erythro-MAPP and D-threo-NMAPPD are added in concentrations of 0; 1; 2; 10; and 20 mM in 5 μl of ethanol resulting in final concentrations of 0; 50; 100; 500; and 1000 μM. Incubation at 37° C. is carried out for 0 and 20 min after establishment of linearity of product formation within this time frame.

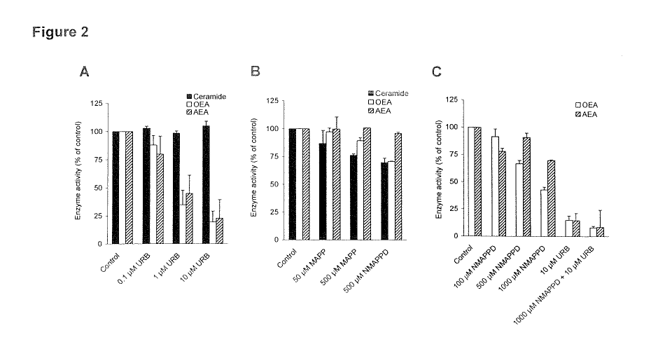

The FAAH inhibitor URB597 inhibited acylethanolamide hydrolysis dose-dependently with approximately 79% inhibition at a concentration of 10 μM (FIG. 2A) while a similar study utilizing liver membrane preparation as enzyme source found complete inhibition of FAAH activity when adding 10 μM of URB597 (20). At the same pH (7.0), the ceramidase inhibitor D-erythro-MAPP inhibited ceramide hydrolysis approximately 24% and OEA hydrolysis by 11% while AEA hydrolysis was unaffected. The same concentration of D-threo-NMAPPD resulted in 30% inhibition of hydrolysis of ceramide as well as OEA (FIG. 2B). D-threo-NMAPPD showed dose-dependent inhibition of OEA hydrolysis, although the compound had relatively low potency (FIG. 2C). Furthermore, an additive inhibitory effect of D-threo-NMAPPD and URB597 on OEA hydrolysis was evident (FIG. 2C). These results indicate inhibition of hydrolysis of OEA by inhibition of an enzyme different from FAAH. This enzyme is suggested to be a ceramidase since D-erythro-MAPP and D-threo-NMAPPD are ceramidase inhibitors.

### Example 2

### ph Dependency of A) OEA Hydrolysis, B) Ceramide Hydrolysis, and C) Anandamide Hydrolysis by Intestinal Protein from (URB597 Treated) Rats

2A: The pH-dependency of OEA hydrolysis without D-erythro-MAPP is assayed using similar conditions as described in example 1A with 50 μg intestinal protein from Sprague Dawley rats (approximately 200 g), URB597 treated (0.3 mg/kg i.p. 1 h prior to anaesthesia) and untreated [4 ml/kg of vehicle (saline/Tween 80/polyethylene glycol; 90:5:5) i.p. 1 h prior to anaesthesia], incubated with 28 μM [1−³H-ethanolamine]OEA (10 dpm/pmol) for 0, 10, 20, and 30 min at 37° C. in a total volume of 200 μl of varying buffers containing 0.9 mM EDTA, 1.5 mg/ml fatty acid-free bovine serum albumin. Alternatively, 25 μg of protein from rat intestinal (jejunum) homogenate in a total volume of 100 μl and 50 μM of ³H-oleoylethanolamide (OEA; 25.000 dpm) was used with or without 10 μM of URB597 (200 μM added in 5 μl of DMSO) and incubated for 0 and 20 min at 37° C. At pH 4.0; 4.5; 5.0; 5.5; 6.0; 6.5; 7.0 100 mM citrate-NaHPO₄ buffer is used, at pH 7.0; 7.5; 8.0; 8.5; 9.0; 9.0 100 mM Tris-HCl is used, and at pH 9.0; 9.5; 10.0 100 mM Na₂CO₃—NaHCO₃ or 100 mM glycin-NaOH is utilized.

2B: The pH-dependency of ceramide hydrolysis without D-erythro-MAPP using similar conditions as described in example 1B with 50 μg intestinal protein from male Sprague Dawley rats (approximately 200 g), URB597 treated (0.3 mg/kg i.p. 1 h prior to anaesthesia) and untreated [4 ml/kg of vehicle (saline/Tween 80/polyethylene glycol; 90:5:5) i.p. 1 h prior to anaesthesia], incubated with 0.5 mM N-[1-¹⁴C]oleoyl-D-erythro sphingosine (500 dpm/nmol) for 0, 15, 30, and 60 min at 37° C. in a total volume of 100 μl of varying buffers containing 8 mM of CHAPS. Alternatively, 25 μg of protein from rat intestinal (jejunum) homogenate in a total volume of 100 μl and 50 μM of ¹⁴C-Octanoyl-D-sphingosine (ceramide; 25.000 dpm) was used with or without 10 μM of URB597 (200 μM added in 5 μl of DMSO) and incubated for 0 and 20 min at 37° C. At pH 4.0; 4.5; 5.0; 5.5; 6.0; 6.5; 7.0 100 mM citrate-NaHPO₄ buffer is used, at pH 7.0; 7.5; 8.0; 8.5; 9.0; 9.5 100 mM Tris-HCl is used, and at pH 9.0; 9.5; 10.0 100 mM Na₂CO₃—NaHCO₃ or 100 mM glycin-NaOH is utilized.

2C: The pH-dependency of anandamide hydrolysis is investigated exactly as described in 2A except for the substrate, which is replaced by 28 μM [1−³H-ethanolamine]anandamide (10 dpm/pmol) or 50 μM of ³H-anandamide (5000 dpm/nmol=25.000 dpm in 100 μl).

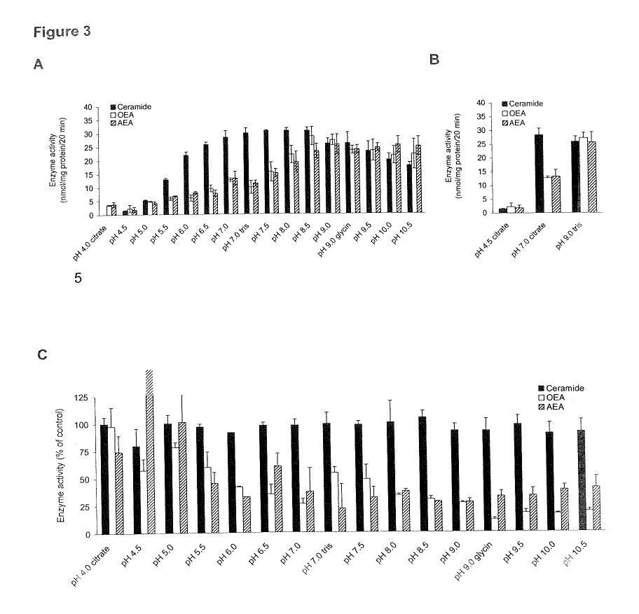

A complete study of pH dependency on rate of hydrolysis of ceramide, OEA and AEA showed a broad peak of hydrolysis of ceramide from pH 7 to pH 9, while both acylethanolamides were hydrolysed to a lesser extent up to pH 9 through 10.5 (FIG. 3A). At selected pH values the experiment was repeated up to 7 times (FIG. 3B). The results show a very similar extent of hydrolysis of OEA and AEA while hydrolysis of ceramide clearly has a separate pH profile, thereby suggestive of the presence of at least two separate enzymes, which are responsible for acylethanolamide hydrolysis and ceramide hydrolysis, respectively. This is substantiated in FIG. 3C showing the degree of hydrolysis of the three separate substrates with the specific FAAH inhibitor URB597 present. Ceramide hydrolysis was completely unaffected by URB597 while hydrolysis of OEA and AEA to varying degrees were inhibited from pH 5.5 to pH 10.5. The lack of complete inhibition of OEA and AEA hydrolysis in the presence of URB597 in rat intestinal homogenate also indicate two or more separate enzymes capable of hydrolysing these acylethanolamides.

In conclusion, the present in vitro data indicate a specific hydrolytic activity distinct from FAAH, capable of hydrolyzing ceramide and OEA and to a lesser degree also AEA in rat intestinal homogenate. This enzymatic activity can be inhibited by ceramidase inhibitors such as D-erythro-MAPP and D-threo-NMAPPD.

### Example 3

### Inhibition of Food Intake in Mice/Rats Following MAPP/NMAPPD Administration

In order to evaluate the effect of a ceramidase inhibitor on food intake—possibly via prolongation of the effect of endogenously produced OEA the following in vivo experiments were designed. Furthermore, co-administration of a sub-maximal dose of OEA along with a ceramidase inhibitor was planned to substantiate the possible mode of action of the compounds of the invention.

3A: Inhibition of food intake in rats following administration of an inhibitor of ceramidase. Thirty male Sprague Dawley rats (6 weeks of age, approximately 190 g, Charles River, Germany) are used following the acclimatization protocol described above. The rats are randomized into weight-matched groups of n=6 then fasted for 24 h with removal of food just before beginning of the dark period. The next day they receive an acute dose of 0.05; 0.2; 0.5; 2; or 5 mg/kg of D-erythro-MAPP or vehicle (saline containing 2.8% ethanol) administered i.v. (4 ml/kg) just prior to the dark period.

A parallel assay measures the acute effect of D-threo-NMAPPD as well as the respective enantiomers and diastereomers of both compounds on food intake in rats following the fasting phase or in the absence of food restriction prior to administration of the test compounds.

3B: Inhibition of food intake in rats following administration of an inhibitor of ceramidase in conjunction with OEA. Thirty male Sprague Dawley rats (6 weeks of age, approximately 190 g, Charles River, Germany) are used following the acclimatization protocol described above. The rats are randomized into weight-matched groups of n=6 then fasted for 24 h with removal of food just before the beginning of the dark period. The next day four of the five groups of animals receive an acute dose of 40 mg/kg of OEA and one group receive the vehicle (5% Tween 80, 5% polyethylene glycol, 90% saline) administered p.o. (4 ml/kg) less than 1 h prior to presentation of the food (onset of darkness). Three of the four OEA-treated groups are also given D-erythro-MAPP 0.5; 2; or 5 mg/kg administered i.v. (4 ml/kg in saline containing 2.8% ethanol) just prior to the dark period.

A similar set up is used for investigations of the acute effect on food intake of D-threo-NMAPPD in combination with OEA as well as the respective enantiomers as well as diastereomers of D-erythro-MAPP and D-threo-NMAPPD.

3C. Furthermore, a study with dietary obese mice was carried out. After 12 weeks on high-fat diet, animals were stratified according to weight. At day −3, animals were randomised in 5 groups (n=9-10 in each group) to participate in one of following drug treatment groups; vehicle (5% Tween 80, 5% polyethylene glycol, 90% saline), OEA 2 mg/kg, OEA 5 mg/kg, D-threo-NMAPPD 30 mg/kg, D-threo-NMAPPD 30 mg/kg+OEA 2 mg/kg (mixed). The animals received one intraperitoneal injection daily (2 ml/kg) of vehicle or drug(s) suspended in vehicle at 2:30 μM. The experiment was preceded by a 3 day run-in period with mock injections and handling to habituate the animals to the injection paradigm. On day 0 the animals were dosed for the first time. All animals were fed high-fat diet ad lib—also during the treatment period. Body weight, food and water intake was measured every other day for the following 14 days depending on the animal's state.

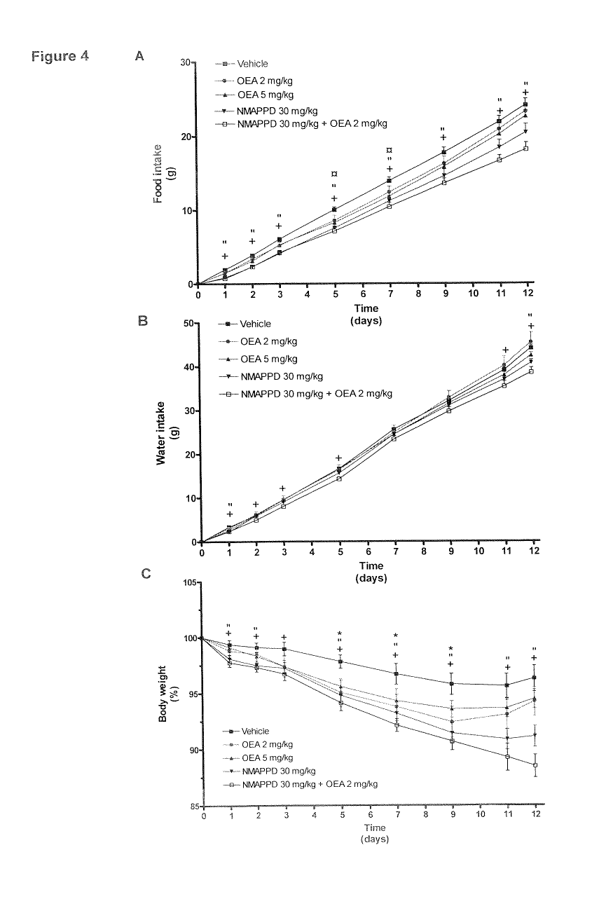

Food intake was consistently and significantly reduced from day 1 of the experiment in both groups receiving the ceramidase inhibitor D-threo-NMAPPD (FIG. 4A). At day 5 and 7 also the group treated with 5 mg/kg of OEA reached significantly reduced food intake. There was a clear tendency of an additive effect of D-threo-NMAPPD and OEA when co-administered as seen from the cumulative food intake of only 18.0±1.0 g at day 12. In comparison, administration of D-threo-NMAPPD resulted in 20.4±1.2 g of food intake and administration of 2 mg/kg of OEA resulted in 23.3±1.0 g, which was not significantly different from food intake of vehicle-treated animals (24.1±0.9 g). Water intake was less affected than food intake by the treatments resulting only in significant decreased water intake of D-threo-NMAPPD-treated animal at day 1 and day 12 while co-administration of D-threo-NMAPPD and OEA resulted in more pronounced reduction of water intake (FIG. 4B). Body weight was significantly affected by the D-threo-NMAPPD treatment resulting in loss of 8.9% of body weight at day 12 while co-administration of D-threo-NMAPPD and OEA resulted in 11.9% loss of body weight compared to 3.7% in the vehicle-treated animals (FIG. 4C). A body weight gain was not seen in control animals due to the daily i.p. injection.

Long-term elevated OEA levels resulting from daily administration of 5 mg/kg of OEA i.p. to diet-induced obese mice as well as rats have previously been reported to result in reduced body weight change from day 2 and on. This was explained by stimulation of lipolysis by activation of PPARalpha (9,22). The present results demonstrate that administration of a ceramidase inhibitor such as D-threo-NMAPPD can indeed inhibit food intake as well as body weight gain in dietary obese mice. Furthermore, it was here demonstrated that the proposed mechanism of action resulting from a putatively prolonged effect of endogenously produced OEA by inhibition of ceramidase can be re-enforced by co-administration of OEA along with D-threo-NMAPPD. These results underline that even a modest in vitro inhibitory effect of D-erythro-MAPP and D-threo-NMAPPD may result in very efficient in vivo inhibitory effect on food intake. This is to some extent surprising, but may be due to different compartmentation of the enzymes (ceramidase in comparison to FAAH) combined with a favourable local accumulation of OEA and/or the ceramidase inhibitor close to the hydrolytic enzyme, ceramidase.

### CITED DOCUMENTS

1. Crowley, V. E. F., Yeo, G. S. H., and O'Rahilly, S. 2002. Obesity therapy: Altering the energy intake-and-expenditure balance sheet. Nature Reviews 1: 276-286.
2. Müller M. J., Mast, M., Asbeck, I., Langnäse, K., and Grund, A. 2001. Prevention of obesity—is it possible? Obesity Reviews 2: 15-28.
3. Chiesi, M., Huppertz, C., and Hofbauer, K. G. 2001. Pharmacotherapy of obesity: targets and perspectives. Trends Pharmacol. Sci. 22: 247-254.
4. Hansen, H. S., Moesgaard, B., Hansen, H. H., and Petersen, G. 2000. N-Acylethanolamines and precursor phospholipids—relation to cell injury. Chem. Phys. Lipids 108: 135-150.
5. Rodriguez de Fonseca, F., Navarro, M., Gómez, R., Escuredo, L., Nava, F., Fu, J., Murillo-Rodríguez, E., Giuffrida, A., LoVerme, J., Gaetani, S. et al. 2001. An anorexic lipid mediator regulated by feeding. Nature 414: 209-212.
6. Lo Verme, J., Gaetani, S., Fu, J., Oveisi, F., Burton, K., and Piomelli, D. 2005. Regulation of food intake by oleoylethanolamide. Cell. Mol. Life Sci. 62: 708-716.
7. Nielsen, M. J., Petersen, G., Astrup, A., and Hansen, H. S. 2004. Food intake is inhibited by oral oleoylethanolamide. J. Lipid Res. 45: 1027-1029.
8. Oveisi, F., Gaetani, S., Eng, K. T.-P., and Piomelli, D. 2004. Oleoylethanolamide inhibits food intake in free-feeding rats after oral administration. Pharmacological Research 49: 461-466.
9. Fu, J., Gaetani, S., Oveisi, F., LoVerme, J., Serrano, A., Rodriguez de Fonseca, F., Rosengarth, A., Luecke, H., Di Ciacomo, B., Tarzia, G. et al. 2003. Oleoylethanolamide regulates feeding and body weight through activation of the nuclear receptor PPARa. Nature 425: 90-93.
10. Sanchis, D., Balada, F., del Mar Grasa, M., Virgili, J., Peinado, J., Monserrat, C., Fernández-López, J. A., Remesar, X., and Alemany, M. 1996. Oleoyl-estrone induces the loss of body fat in rats. Int. J. Obes. 20: 588-594.
11. Alemany, M., Fernandez-Lopez, J. A., Petrobelli, A., Granada, M., Foz, M., and Remesar, X. 2003. Weightloss in a patient with morbid obesity under treatment with oleoyl-estrone. Med. Clin. (Barc.) 121: 496-499.
12. Ueda, N., Kurahashi Y., Yamamoto S., and Tokunaga T. 1995. Partial purification and characterization of the porcine brain enzyme hydrolyzing and synthesizing anandamide. J. Biol. Chem. 270: 23823-23827.
13. Ueda, N., Yamanaka, K., and Yamamoto, S. 2001. Purification and characterization of an acid amidase selective for N-palmitoylethanolamine, a putative endogenous anti-inflammatory substance. J. Biol. Chem. 276: 35552-35557.
14. Fegley D., Gaetani S., Duranti A., Tontini A. Mor M., Tarzia G., and Piomelli D. 2005. Characterization of the fatty acid amide hydrolase inhibitor cyclohexyl carbamic acid 3′-carbamoyl-biphenyl-3-yl ester (URB597): Effects on anandamide and oleoylethanolamide deactivation. J. Pharmacol. Exp. Ther. 313: 352-358.
15. Sugita, M., Williams, M., Dulaney, J., and Moser, H.1975. Ceramidase and ceramide synthesis in human kidney and cerebellum. Description of a new alkaline ceramidase. Biochim. Biophys. Acta 398:125-133.
16. Olsson, M., Duan, R. D., Ohisson, L., and Nilsson, A. 2004. Rat intestinal ceramidase: purification, properties, and physiological relevance. Am. J. Physiol. Gastrointest. Liver Physiol. 287: G929-G937.
17. Bielawska, A., Linardic, C. M., and Hannun, Y. A. 1992. Ceramide-mediated biology. J. Biol. Chem. 267:18493-18497.
18. Bauer, R. and Remiger, P. 1989. TLC and HPLC analysis of alkamides in Echinacea drugs. Planta Medica 55: 367-371.
19. Abadji, V., Lin, S., Taha, G., Griffin, G., Stevenson, L. A., Pertwee, R. G., and Makriyannis, A. 1994. (R)-methanandamide: a chiral novel anandamide possessing higher potency and metabolic stability. J. Med. Chem. 37:1889-1893.
20. Sun, Y. X., Tsuboi, K., Zhao, L. Y., Okamoto, Y., Lambert, D. M., and Ueda, N. 2005. Involvement of N-acylethanolamine-hydrolyzing acid amidase in the degradation of anandamide and other N-acylethanolamines in macrophages. Biochim. Biophys. Acta. 1736: 211-220.
21. Lichtman, A. H., Hawkins, E. G., Griffin, G., and Cravatt, B. F. 2002. Pharmacological activity of fatty acid amides is regulated, but not mediated, by fatty acid amide hydrolase in vivo. J. Pharmacol. Exp. Ther. 302: 73-79.
22. Guzmán, M., Lo Verme, J., Fu, J., Oveisi, F., Blázquez, C., and Piomelli, D. 2003. Oleoylethanolamide stimulates lipolysis by activating the nuclear receptor peroxisome proliferator-activated receptor α (PPAR-α). J. Biol. Chem. 279: 27849-27854.

1.-11. (canceled)

12. A method for suppressing appetite or for the treatment of overweight, obesity and/or type II diabetes, the method comprising administering to a mammal in need thereof an effective amount of a compound, wherein said compound is an appetite suppressing or satiety inducing agent with the structure of formula I: 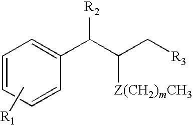
wherein m is an integer ranging from 0 to 22;
Z is a member selected from —C(O)N(R₄)—; —(R₄)NC(O)—; —OC(O)—; —
(O)CO—; O; NR₄; and S; and
R₁, R₂, R₃, and R₄ are independently selected from the group consisting of substituted or unsubstituted alkyl, hydrogen, NO₂, OH, methoxy, chlorine, bromine, fluorine, substituted or unsubstituted C₁-C6 alkyl, substituted or unsubstituted lower (C₁-C₆) acyl, ether, homoalkyl, and aryl, and
from 0 to 12 hydrogen atoms of the compound are substituted by a methyl group, a double bond, or a triple bond.

13.-44. (canceled)

45. A solid composition for use as a medicament comprising a ceramidase inhibitor with the formula I: 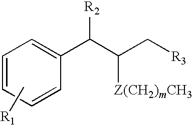
wherein m is an integer ranging from 0 to 22;
Z is a member selected from —C(O)N(R₄)—; —(R₄)NC(O)—; —OC(O)—; —
(O)CO—; O; NR₄; and S; and
R₁, R₂, R₃, and R₄ are independently selected from the group consisting of substituted or unsubstituted alkyl, hydrogen, NO₂, OH, methoxy, chlorine, bromine, fluorine, substituted or unsubstituted C₁-C6 alkyl, substituted or unsubstituted lower (C₁-C₆) acyl, ether, homoalkyl, and aryl, and
from 0 to 12 hydrogen atoms of the compound are substituted by a methyl group, a double bond, or a triple bond, and further comprising one or more appetite suppressing compounds with the formula: 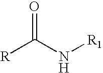
wherein R—C═O is derived from a natural or synthetic fatty acid and R₁ is i) a branched or unbranched, saturated or unsaturated, substituted or unsubstituted chain of from 1 to 30 carbon atoms, which optionally is substituted with one or more hydroxy groups, which may be primary, secondary or tertiary, or ii) an N-terminal amino acid or peptide residue.

46. A composition according to claim 45, having a form selected from the group consisting of a tablet, capsule, sachet, powder and granules.

47. A method as claimed in claim 12, wherein said compound is of the formula II: 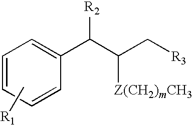
wherein m is an integer ranging from 6 to 18;
R₁, R₂, R₃, and R₄ are independently selected from the group consisting of substituted or unsubstituted alkyl, hydrogen, NO₂, OH, methoxy, chlorine, bromine, fluorine, substituted or unsubstituted C₁-C6 alkyl, substituted or unsubstituted lower (C₁-C₆) acyl, ether, homoalkyl, and aryl, and
from 0 to 12 hydrogen atoms of the compound are substituted by a methyl group, a double bond, or a triple bond.

48. A method as claimed in claim 12, further comprising repeating said dosage until a cosmetically beneficial loss of body weight has occurred.

49. A method as claimed in claim 12, wherein said compound is administered in a dosage sufficient to effect a reduction of fat tissue mass/lean mass in said mammal.

50. A method as claimed in claim 12, wherein said compound is formulated as a dietary supplement.

51. A method as claimed in claim 12, wherein said mammal is a human or domestic animal.

52. A method as claimed in claim 47, wherein an acyl group at any of R₁, R₂, R₃, and R₄ is an acyl derivative of a C₂-C₄ acid.

53. A method as claimed in claim 52, wherein the fatty acid moiety is selected from the group consisting of lauric acid, myristic acid, and palmitic acid.

54. A method as claimed in claim 52, wherein the fatty acid moiety is selected from the group consisting of N-lauroyl-2-amino-1-phenyl-1-propanol, N-lauroyl-2-amino-1-(4′-nitrophenyl)-1,3-propandiol, N-myristoyl-2-amino-1-phenyl-1-propanol, N-myristoyl-2-amino-1-(4′-nitrophenyl)-1,3-propandiol, N-palmitoyl-2-amino-1-phenyl-1-propanol, and N-palmitoyl-2-amino-1-(4′-nitrophenyl)-1,3-propandiol.

55. A method as claimed in claim 47, wherein m is an integer between 10 to 14; and members R₁, R₂, R₃, and R₄ are independently selected from the group consisting of substituted or unsubstituted alkyl, hydrogen, NO₂, OH, methoxy, chlorine, bromine and fluorine.

56. A method as claimed in claim 12, wherein said compound is an N-acyl-phenylaminoalcohol selected from (1S,2R)-D-erythro-N-myristoyl-2-amino-1-phenyl-propanol and (1R,2R) D-threo-N-myristoyl-2-amino-1-(4′-nitrophenyl)-1,3-propandiol.

57. A method as claimed in claim 12, wherein the compound is administered in an amount in a range selected from ranges consisting of about 0.1 μg/kg to about 1 μg/kg body weight; about 1 μg/kg to 10 μg/kg body weight; about 10 μg/kg to about 0.1 mg/kg body weight; about 0.1 mg/kg to about 1 mg/kg; about 1 mg/kg to about 10 mg/kg body weight; about 10 mg/kg to about 50 mg/kg body weight; about 50 mg/kg to about 100 mg/kg body weight; about 100 mg/kg to about 250 mg/kg body weight; about 250 mg/kg to about 500 mg/kg body weight; and about 500 mg/kg to about 1 g/kg body weight.

58. A method as claimed in claim 12, wherein said composition further comprises one or more appetite suppressing compound with the structure: 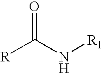
wherein
R—C═O is derived from a natural or synthetic fatty acid, and R1 is i) a branched or unbranched, saturated or unsaturated, substituted or unsubstituted chain of from 1 to 30 carbon atoms, which optionally is substituted with one or more hydroxy groups, which may be primary, secondary or tertiary, or ii) an N-terminal amino acid or peptide residue.

59. A method as claimed in claim 58, wherein the fatty acid of said further one or more appetite suppressing compounds is a branched or unbranched, cyclic or acyclic, saturated or unsaturated, substituted or unsubstituted chain of from 3 to 28 carbon atoms.

60. A method as claimed in claim 59, wherein the fatty acid of said further one or more appetite suppressing compound has a chain of from 14 to 22 carbon atoms.

61. A method as claimed in claim 58, wherein the chain of carbon atoms has from 0 to 3 double bonds.

62. A method as claimed in claim 58, wherein the chain of carbon atoms has from 1 to 4 triple bonds.

63. A method as claimed in claim 58, wherein R₁ is an alk amine optionally substituted by a group selected from among one or more hydroxy groups, a sphingoid base, an amino acid and a peptide, wherein alk is alkyl or alkenyl.

64. A method as claimed in claim 58, wherein the compound is an N-acylalkanolamine.

65. A method as claimed in claim 58, wherein the compound is an N-acylethanolamine.

66. A method as claimed in claim 58, wherein the compound is a naturally occurring N-acylethanolamine.

67. A method as claimed in claim 58, wherein the compound is selected from the group consisting of N-oleoylethanolamine, N-palmitoylethanolamine, N-linoleoylethanolamine, N-[alpha]-linolenoylethanolamine, N-[gamma]-linolenoylethanolamine, N— acylpropan-1-ol-2-amine, N— oleoylpropan-1-ol-2-amine and N-arachidonoylpropan-1-ol-2-amine.
